(19) United States
(12) Patent Application Publication
(10) Pub. No.: US 2014/0234935 A1
(43) Pub. Date: Aug. 21, 2014

(54) METHOD FOR PRODUCING ETHANOL USING CELLULOSIC BIOMASS AS RAW MATERIAL

(71) Applicant: KAWASAKI JUKOGYO KABUSHIKI KAISHA, Kobe-shi, Hyogo (JP)

(72) Inventors: Hiromasa Kusuda, Kobe-shi (JP); Noriaki Izumi, Kobe-shi (JP); Hironori Taijiri, Kobe-shi (JP); Shoji Tsujita, Itami-shi (JP); Takashi Nishino, Suita-shi (JP)

(73) Assignee: KAWASAKI JUKOGYO KABUSHIKI KAISHA, Kobe-shi, Hyogo (JP)

(21) Appl. No.: 14/346,707

(22) PCT Filed: Sep. 24, 2012
(86) PCT No.: PCT/JP2012/006048

§ 371 Date: Mar. 21, 2014

(30) Foreign Application Priority Data
Sep. 30, 2011 (JP) 2011-218034

(51) Int. Cl. C12P 7/10

(52) U.S. Cl. CPC C12P 7/10; C12P 2203/00
USPC 435/165

(57) ABSTRACT

According to the method of the present invention, a cellulosic biomass slurry whose concentration of cellulosic biomass between 1% and 5% by mass is hydrothermally treated at a temperature of between 140° C. and 200° C. a pressure of between 1 MPa and 5 MPa to saccharify/decompose hemicellulose into C5 sugars. Then, a dewatered cake obtained after the hydrothermal treatment is slurried and has a solid concentration of between 1% and 5% by mass, and the slurry is hydrothermally treated at a temperature of between 240° C. and 300° C. and a pressure of between 4 MPa and 10 MPa to saccharify/decompose cellulose into C6 sugars. A saccharified solution is concentrated by a concentration device such as a reverse osmosis membrane device so that the concentration of sugars is 10% by mass or higher, and is then subjected to alcoholic fermentation.

### TECHNICAL FIELD

The present invention relates to a method for producing ethanol (bioethanol) by alcoholic fermentation of sugars produced by hydrolyzing cellulosic biomass in a supercritical or subcritical state.

### BACKGROUND ART

As part of utilizing biomass energy, attempts have been made to decompose cellulose or hemicellulose as main components of plants to obtain ethanol. The thus obtained ethanol is planned to be mainly used as fuel such as part of automotive fuel or a gasoline alternative.

The main components of plants include cellulose (a polymer of glucose as a C6 sugar containing 6 carbon atoms), hemicellulose (a polymer of a C5 sugar containing 5 carbon atoms and a C6 sugar), lignin, and starch. Ethanol is produced by fermentation action of microorganisms, such as yeast, using, as a raw material, sugars such as C5 sugars, C6 sugars, and oligosaccharides as complexes of them.

The industrial use of the following three methods is being contemplated to decompose cellulosic biomass such as cellulose or hemicellulose into sugars: 1) a hydrolysis method utilizing the oxidation power of a strong acid such as sulfuric acid; 2) an enzymatic decomposition method; and 3) a method utilizing the oxidation power of supercritical water or subcritical water. However, it is difficult to practically use the acid decomposition method 1) from an economical viewpoint. This is because an added acid acts as an inhibitor of yeast fermentation, and therefore absolutely needs to be neutralized after decomposition of cellulose or hemicellulose into sugars and before alcoholic fermentation of the sugars, and the neutralization treatment is costly. The enzymatic decomposition method 2) can be performed at ordinary temperature and constant pressure, but no effective enzyme has been found. Even if an effective enzyme is found, it is expected that the production cost of the enzyme will be expensive. Therefore, from an economical viewpoint, there seems to be no prospect for actually using the enzymatic decomposition method on an industrial scale.

Patent Literature 1 discloses, as a method for hydrolyzing cellulosic biomass into sugars with supercritical water or subcritical water, a method for producing a water-insoluble polysaccharide by bringing a cellulose powder into contact with pressurized hot water at 240 to 340° C. to hydrolyze cellulose. Patent Literature 2 discloses a method in which biomass cut into small pieces is hydrolyzed with hot water pressurized to a saturated water vapor pressure or higher at 140 to 230° C. for a predetermined time to decompose/extract hemicellulose, and is then hydrolyzed with pressurized hot water heated to a decomposition temperature of cellulose or higher to decompose/extract cellulose. Patent Literature 3 discloses a method for producing glucose and/or a water-soluble cello-oligosaccharide, in which cellulose having an average degree of polymerization of 100 or higher is subjected to a contact reaction with supercritical water or subcritical water at a temperature of 250° C. or higher but 450° C. or lower and a pressure of 15 MPa or higher but 450 MPa or lower for 0.01 second or longer but 5 seconds or shorter, and is then cooled and hydrolyzed by contact with subcritical water at a temperature of 250° C. or higher but 350° C. or lower and a pressure of 15 MPa or higher but 450 MPa or lower for 1 second or longer but 10 minutes or shorter.

Patent Literature 4 discloses a sugar production method capable of not only obtaining sugars from wood-based biomass in high yield with high efficiency but also separately recovering sugars containing C5 sugars and C6 sugars and sugars containing C6 sugars. The sugar production method disclosed in Patent Literature 4 includes: a first slurry heating step (S1) of heat-treating a slurry prepared by adding high temperature and high pressure water to wood-based biomass; a first separation step (S2) of separating the heat-treated slurry into a liquid component and a solid component; a second slurry heating step (S3) of adding water to the separated solid component to prepare a slurry and heat-treating the slurry; a second separation step (S4) of separating the heat-treated slurry into a liquid component and a solid component; and a useful component obtaining step (S5) of removing water from the separated liquid component to obtain sugars, wherein in the useful component obtaining step (S5), in addition to obtaining sugars, removal of water from the liquid component separated in the first separation step (S2) is further performed to obtain sugars.

### CITATION LIST

### Patent Literatures

PTL 1: Japanese Laid-Open Patent Application Publication No. 2000-186102
PTL 2: Japanese Laid-Open Patent Application Publication No. 2002-59118
PTL 3: Japanese Laid-Open Patent Application Publication No. 2003-212888
PTL 4: Japanese Laid-open Patent Application Publication No. 2010-81855

### SUMMARY OF INVENTION

### Technical Problem

The conventional art, in which cellulosic biomass is hydrolyzed into sugars with supercritical water or subcritical water, can save energy by increasing the concentration of biomass (solid concentration) in a cellulosic biomass slurry to be hydrothermally treated, because a larger amount of biomass can be treated.

Here, in a conventional hydrolysis method, biomass is subjected to hydrothermal treatment (first hydrothermal treatment) to hydrolyze hemicellulose in the biomass into C5 sugars, a residue is dewatered, and a solid matter (solid residue) is reslurried and subjected to hydrothermal treatment (second hydrothermal treatment) under severer conditions to hydrolyze cellulose in the biomass into C6 sugars. However, about 10% of the C5 sugars produced by the first hydrothermal treatment remain in a residue obtained by dewatering treatment after the first hydrothermal treatment. The C5 sugars are oxidized by the second hydrothermal treatment to an inhibitor, such as organic acids, that inhibits alcoholic fermentation performed in a subsequent fermentation step.

Therefore, the amount of C5 sugars that will remain in a residue obtained after the first hydrothermal treatment is increased by increasing the concentration of biomass in a cellulosic biomass slurry for improving the efficiency of hydrolysis. As a result, the loss of C5 sugars is increased and a reduction in the efficiency of alcoholic fermentation is also caused. An increase in the concentration of the slurry reduces the fluidity of the slurry, which makes it difficult to transfer the slurry through piping. Further, heat conductivity in an indirect heat exchanger is also reduced.

It is an object of the present invention to prevent the loss of C5 sugars and to suppress the formation of a fermentation inhibitor in the step of saccharifying hemicellulose and cellulose, in a method for producing ethanol by alcoholic fermentation of a saccharified solution obtained by separately hydrolyzing hemicellulose and cellulose in cellulosic biomass.

### Solution to Problem

The present inventors have intensively studied, and as a result, have found that C5 sugars are less likely to remain in a dewatered cake as a residue of solid-liquid separation of a slurry after hydrothermal treatment when the concentration (solid concentration) of cellulosic biomass to be subjected to hydrothermal treatment for hydrolyzing hemicellulose is kept low, which has led to the completion of the present invention.

More specifically, the present invention provides a method for producing ethanol using cellulosic biomass as a raw material, characterized by including:

a first hydrolytic saccharification step of hydrothermally treating a slurry of cellulosic biomass whose solid concentration is 1% by mass or higher but 5% by mass or lower at a temperature of 140° C. or higher but 200° C. or lower and a pressure of 1 MPa or higher but 5 MPa or lower to saccharify/decompose hemicellulose contained in the cellulosic biomass into C5 sugars;

a first solid-liquid separation step of subjecting the slurry after the first hydrolytic saccharification step to solid-liquid separation;

a reslurrying step of adding water to a dewatered cake obtained in the first solid-liquid separation step to prepare a slurry having a solid concentration of 1% by mass or higher but 5% by mass or lower;

a second hydrolytic saccharification step of hydrothermally treating the slurry obtained in the reslurrying step at a temperature of 240° C. or higher but 300° C. or lower and

a pressure of 4 MPa or higher but 10 MPa or lower to saccharify/decompose cellulose contained in the cellulosic biomass into C6 sugars;

a second solid-liquid separation step of subjecting the slurry after the second hydrolytic saccharification step to solid-liquid separation;

a concentration step of concentrating a C5 saccharified solution obtained in the first solid-liquid separation step and a C6 saccharified solution obtained in the second solid-liquid separation step so that the concentration of sugars is 10% by mass or higher;

a fermentation step of subjecting a concentrated saccharified solution after the concentration step to alcoholic fermentation; and

a distillation step of distilling a fermented liquid obtained in the fermentation step to concentrate ethanol.

By adjusting the solid concentration (concentration of cellulosic biomass) to 1% by mass or higher but 5% by mass or lower, C5 sugars are less likely to remain in a dewatered cake obtained by subjecting the slurry after the first hydrolytic saccharification step to solid-liquid separation. By adjusting the concentration (solid concentration) of the slurry obtained by adding water to the dewatered cake to be subjected to the second hydrolytic saccharification step to 1% by mass or higher but 5% by mass or lower, C6 sugars are also less likely to remain in a dewatered cake obtained by subjecting the slurry after the second hydrolytic saccharification step to solid-liquid separation.

By adjusting the concentration (solid concentration) of each of the slurry to be subjected to the first hydrolytic saccharification step and the slurry to be subjected to the second hydrolytic saccharification step to 1% by mass or higher but 5% by mass or lower, it is possible to increase the fluidity of the slurry and therefore to easily transfer the slurry through piping. Further, it is possible to improve heat transfer to the slurry in an indirect heat exchanger.

Here, C5 sugars and C6 sugars that will remain in dewatered slurry can be reduced by adjusting the concentrations (solid concentrations) of the slurry to be subjected to the first hydrolytic saccharification step and of the slurry to be subjected to the second hydrolytic saccharification step to 1% by mass or higher but 5% by mass or lower and 1% by mass or higher but 5% by mass or lower, respectively, but this also reduces the concentration (sugar concentration) of a saccharified solution obtained by the first hydrolytic saccharification step and the second hydrolytic saccharification step. As a result, the efficiency of alcoholic fermentation in the subsequent fermentation step is reduced.

However, in the ethanol production method according to the present invention, the saccharified solution is concentrated by a concentrating device such as a reverse osmosis membrane (RO membrane) device before alcoholic fermentation so that the concentration of sugars (total concentration of C5 sugars and C6 sugars) in the saccharified solution is 10% by mass or higher. This makes it possible to keep the concentration of sugars at a level suitable for the subsequent fermentation step to prevent a reduction in the efficiency of alcoholic fermentation.

It is preferred that the first solid-liquid separation step is a step of subjecting the slurry after the first hydrolytic saccharification step to solid-liquid separation and washing a resulting dewatered cake with water and then further subjecting the cake to solid-liquid separation, and that water separated after washing the dewatered cake with water in the first solid-liquid separation step is recovered and subjected to the concentration step.

By washing a dewatered cake obtained from the slurry after the first hydrolytic saccharification step with water, recovering separated water, and subjecting the separated water to the concentration step, it is possible to recover C5 sugars remaining in the dewatered cake.

It is also preferred that the second solid-liquid separation step is a step of subjecting the slurry after the second hydrolytic saccharification step to solid-liquid separation and washing a resulting dewatered cake with water and then further subjecting the cake to solid-liquid separation, and that

water separated after washing the dewatered cake with water in the second solid-liquid separation step is recovered and subjected to the concentration step.

By subjecting the slurry after the second hydrolytic saccharification step to solid-liquid separation and washing a resulting dewatered cake with water and further subjecting the cake to solid-liquid separation in the second solid-liquid separation step and then by recovering separated water and subjecting the separated water to the concentration step, it is possible to recover C6 sugars remaining in the dewatered cake.

The water separated after washing the dewatered cake with water in the first solid-liquid separation step and the water separated after washing the dewatered cake with water in the second solid-liquid separation step may be mixed with the C5 saccharified solution obtained in the first solid-liquid separation step and the C6 saccharified solution obtained in the second solid-liquid separation step and then subjected to the concentration step, or may be subjected to the concentration step separately. However, from the viewpoint of reducing operation time, a mixed liquid of all the saccharified solutions and the washing liquid is preferably subjected to the concentration step.

It is preferred that before the concentration step, the C5 saccharified solution and the C6 saccharified solution are subjected to activated carbon adsorption treatment.

It is preferred that before the C5 saccharified solution and the C6 saccharified solution are concentrated by a reverse osmosis membrane device, a fine solid matter is removed by a microfiltration membrane device (MF membrane device). However, there is a case where a saccharified solution of cellulosic biomass contains an organic matter such as lignin or an inorganic deposit. When such a saccharified solution containing an organic matter or an inorganic deposit is supplied to a reverse osmosis membrane device, an RO membrane is likely to be clogged with the organic matter or the inorganic deposit. Therefore, before the concentration step, the saccharified solution is subjected to activated carbon adsorption treatment to remove an organic matter or an inorganic deposit contained in the saccharified solution so that the clogging of an RO membrane can be prevented.

The C5 saccharified solution and the C6 saccharified solution to be subjected to activated carbon adsorption treatment also include washing water used to wash the dewatered cake obtained from the slurry after the first hydrolytic saccharification step and/or washing water used to wash the dewatered cake obtained from the slurry after the second hydrolytic saccharification step and the C5 saccharified solution and the C6 saccharified solution mixed with the washing water.

The C5 saccharified solution and the C6 saccharified solution concentrated before the fermentation step are preferably subjected to neutralization treatment.

The saccharified solution contains an organic acid, such as acetic acid or lactic acid, formed by hydrolysis of hemicellulose or cellulose. Therefore, the saccharified solution is often acidic with a pH of about 2 to 4. When the saccharified solution is concentrated and directly subjected to the fermentation step, the pH of the saccharified solution is low and is not suitable for ethanol fermentation. Therefore, before the fermentation step, the saccharified solution is preferably neutralized to adjust its pH to about 4.0 to 6.0. For the neutralization treatment is preferably used an alkaline agent, such as caustic soda or hydrated lime, that does not decompose components contained in the saccharified solution or does not inhibit the fermentation step.

The C5 saccharified solution and the C6 saccharified solution to be subjected to neutralization treatment also include washing water used to wash the dewatered cake obtained from the slurry after the first hydrolytic saccharification step and/or washing water used to wash the dewatered cake obtained from the slurry after the second hydrolytic saccharification step and the C5 saccharified solution and the C6 saccharified solution mixed with the washing water.

The foregoing objects, other objects, characteristics, and advantages of the present invention will be made clear from the detailed description of preferred embodiments given below with reference to the attached drawings.

### Advantageous Effects of Invention

According to the ethanol production method of the present invention, it is possible to make the most of C5 sugars and C6 sugars obtained by hydrolysis of hemicellulose and cellulose and to maintain the efficiency of alcoholic fermentation.

### BRIEF DESCRIPTION OF DRAWINGS

FIG. 1 shows a schematic flow chart illustrating Embodiment 1 of the present invention.

FIG. 2 shows a schematic flow chart illustrating Embodiment 2 of the present invention.

FIG. 3 shows a schematic flow chart illustrating Embodiment 3 of the present invention.

FIG. 4 shows a schematic flow chart illustrating Embodiment 4 of the present invention.

FIG. 5 shows a schematic flow chart illustrating Embodiment 5 of the present invention.

### DESCRIPTION OF EMBODIMENTS

Embodiments of the present invention will be described with appropriate reference to the drawings. The present invention is not limited to the description given below.

### Embodiment 1

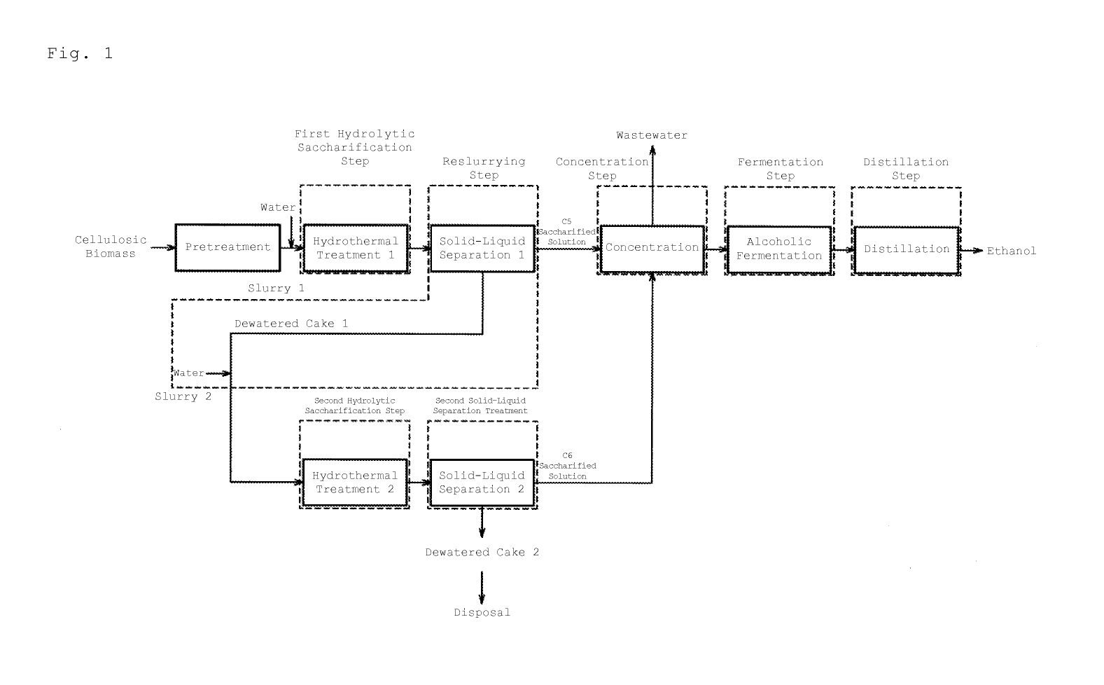

FIG. 1 shows a schematic flow chart illustrating Embodiment 1 of the present invention. First, as pretreatment, cellulosic biomass (e.g., plant-based biomass such as bagasse, sugar beet pulp, or straws) is crushed into small pieces of several millimeters or less. After crushing, water is added to prepare a slurry 1 having a solid concentration of 1% by mass or higher but 5% by mass or lower. The slurry 1 has a low solid concentration, and therefore has high fluidity and is more easily transferred through piping as compared to the conventional art.

(First Hydrolytic Saccharification Step)

Then, the slurry 1 having a solid concentration of 1% by mass or higher but 5% by mass or lower is hydrothermally treated (hydrothermal treatment 1) at a temperature of 140° C. or higher but 200° C. or lower and a pressure of 1 MPa or higher but 5 MPa or lower. The hydrothermal treatment 1 is performed by, for example, applying heat and pressure to the slurry in an indirect heating-type pressure vessel. Hemicellulose in the cellulosic biomass is hydrolyzed into C5 sugars by the hydrothermal treatment 1. At this time, heat conductivity in the indirect heating-type pressure vessel is higher as compared to the conventional art due to high fluidity of the slurry 1.

(First Solid-Liquid Separation Step)

The slurry 1 subjected to the hydrothermal treatment 1 is then subjected to solid-liquid separation (solid-liquid separation 1) by a solid-liquid separator such as a drum filter, a belt filter, a disc filter, or a filter press and thus separated into a C5 saccharified solution and a dewatered cake 1. The C5 saccharified solution is supplied to a subsequent concentration step. At this time, in the present invention, since the solid concentration of the slurry 1 to be hydrothermally treated is lower than that of the slurry to be treated by a conventional hemicellulose hydrolysis method, C5 sugars are less likely to remain in the dewatered cake 1.

(Reslurrying Step)

The dewatered cake 1 is slurried by adding water to prepare a slurry 2 having a solid concentration of 1% by mass or higher but 5% by mass or lower.

(Second Hydrolytic Saccharification Step)

The slurry 2 is hydrothermally treated (hydrothermal treatment 2) at a temperature of 240° C. or higher but 300° C. or lower and a pressure of 4 MPa or higher but 10 MPa or lower in the same manner as in the hydrothermal treatment 1. Cellulose in the cellulosic biomass is hydrolyzed into C6 sugars by the hydrothermal treatment 2. At this time, heat conductivity in the indirect heating-type pressure vessel is higher as compared to the conventional art due to high fluidity of the slurry 2.

In the present invention, the amount of C5 sugars remaining in the dewatered cake 1 is small, and therefore the amount of an alcoholic fermentation inhibitor, such as organic acids, formed by the hydrothermal treatment 2 is smaller as compared to the conventional art.

(Second Solid-Liquid Separation Step)

The slurry 2 subjected to the hydrothermal treatment 2 is subjected to solid-liquid separation (solid-liquid separation 2) by a solid-liquid separator such as a drum filter, a belt filter, a disc filter, or a filter press and thus separated into a C6 saccharified solution and a dewatered cake 2. The C6 saccharified solution is supplied to a subsequent concentration step. The dewatered cake 2 is appropriately disposed of outside the system.

(Concentration Step)

The C5 saccharified solution and the C6 saccharified solution are concentrated by a concentration device such as an RO membrane device so that the concentration of sugars is 10% by mass or higher. When an RO membrane device is used as the concentration device, the C5 saccharified solution and the C6 saccharified solution may be concentrated by the RO membrane device separately from each other, or may be mixed together and then concentrated by the RO membrane device. The concentration of sugars after concentration varies depending on the performance of the RO membrane device, but is preferably higher. The concentration of sugars after concentration is set to about 10% by mass to 50% by mass from a practical viewpoint. In order to prevent the clogging of an RO membrane of the RO membrane device, a solid matter is preferably removed from the C5 saccharified solution and the C6 saccharified solution by, for example, an MF membrane device. Water separated from the saccharified solution by the RO membrane device is appropriately discharged to the outside of the system.

(Fermentation Step)

Then, the concentrated saccharified solution is converted into ethanol by yeast in a fermentation step. The fermentation step can be performed by a publicly known fermentation method. C5 sugars and C6 sugars contained in the saccharified solution are converted into ethanol by the fermentation step.

(Distillation Step)

Then, an alcohol-fermented liquid after the fermentation step is distilled so that ethanol is concentrated. A distillate obtained in the distillation step contains no solid matter and no components other than ethanol. The distillation step can be performed by a distillation method publicly known as a distilled liquor production method.

### Embodiment 2

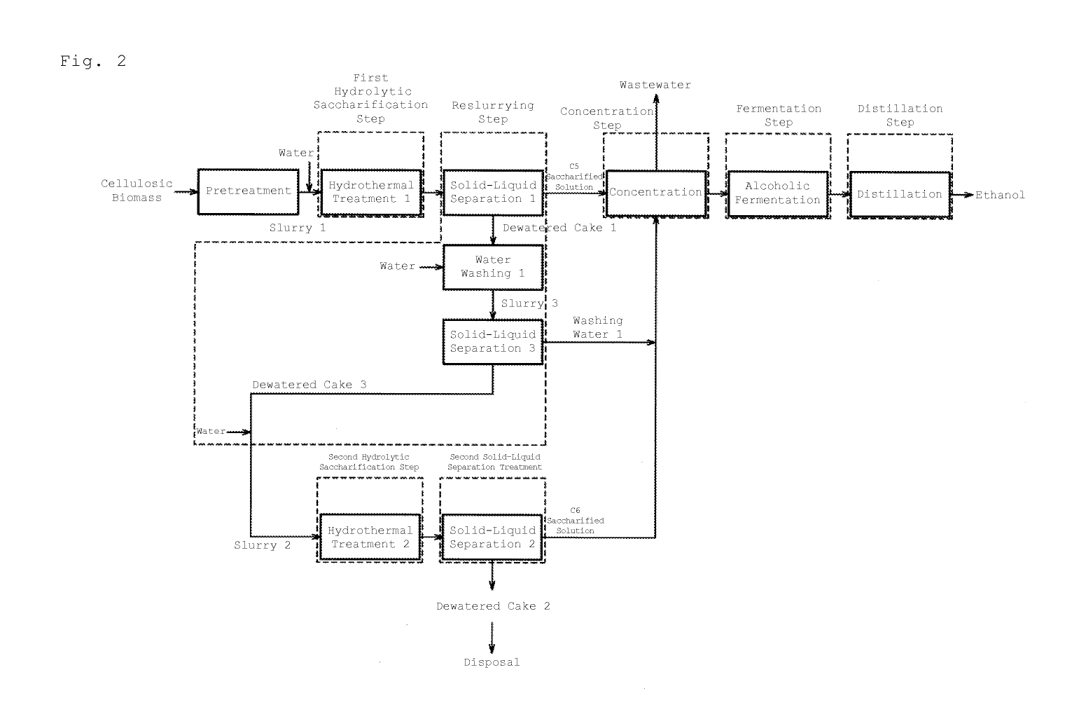

FIG. 2 shows a schematic flow chart illustrating Embodiment 2 of the present invention. A basic flow of this embodiment is the same as that of Embodiment 1, and therefore only the differences from Embodiment 1 will be described here. The same components as in Embodiment 1 are expressed by the same terms as used in Embodiment 1.

This embodiment is different from Embodiment 1 in that water washing treatment 1 and solid-liquid separation treatment 3 are additionally performed before a dewatered cake 1 obtained by solid-liquid separation 1 is subjected to hydrothermal treatment 2. That is, in this embodiment, a dewatered cake 1 obtained by solid-liquid separation 1 is washed with water (water washing 1). By doing so, the dewatered cake 1 is reslurried to prepare a slurry 3. The slurry 3 is subjected to solid-liquid separation (solid-liquid separation 3) in the same manner as in the solid-liquid separation 1 and thus separated into washing water 1 and a dewatered cake 3. The present invention is characterized in that the amount of C5 sugars remaining in the dewatered cake 1 is small. However, according to this embodiment, C5 sugars slightly remaining in the dewatered cake 1 can be recovered maximally by the water washing 1 and supplied to the fermentation step.

The washing water 1 in which C5 sugars are dissolved is mixed with a C6 saccharified solution obtained by solid-liquid separation 2 and then concentrated by an RO membrane device so that the concentration of sugars is 10% by mass or higher. On the other hand, the dewatered cake 3 is slurried by adding water to prepare a slurry 2 having a solid concentration (cellulosic biomass concentration) of 1% by mass or higher but 5% by mass or lower.

### Embodiment 3

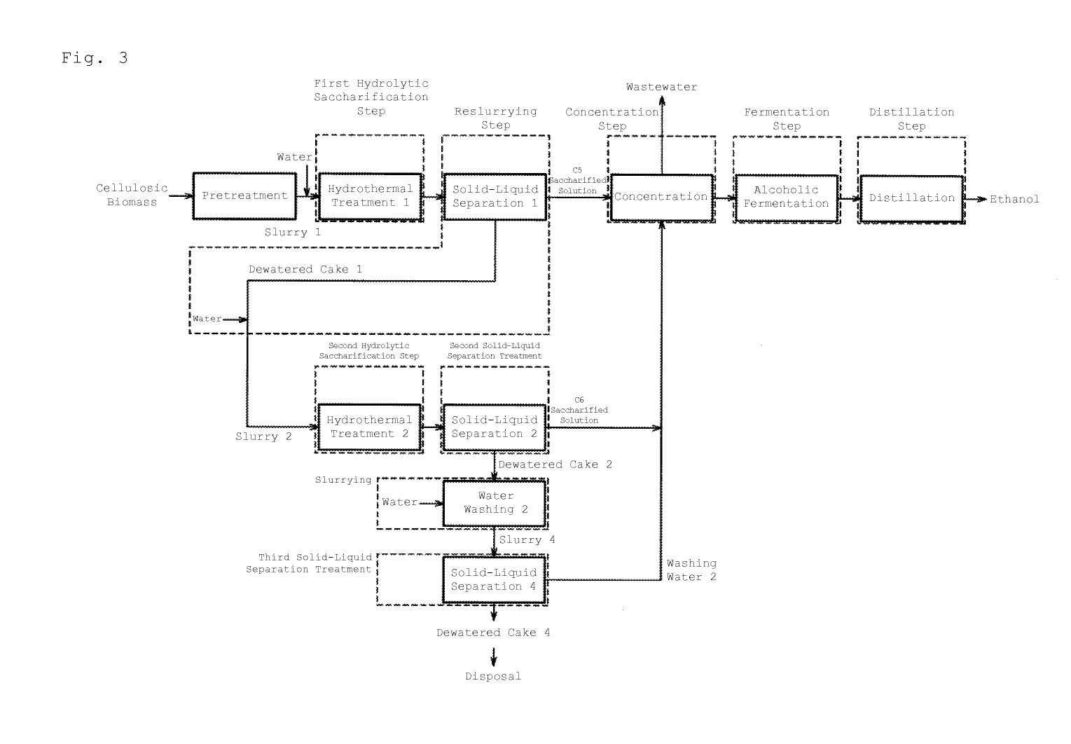

FIG. 3 shows a schematic flow chart illustrating Embodiment 3 of the present invention. A basic flow of this embodiment is the same as that of Embodiment 1, and therefore only the differences from Embodiment 1 will be described here. The same components as in Embodiment 1 are expressed by the same terms as used in Embodiment 1.

This embodiment is different from Embodiment 1 in that water washing treatment 2 and solid-liquid separation treatment 4 are additionally performed on a dewatered cake 2 obtained by solid-liquid separation 2, and washing water 2 obtained by the solid-liquid separation 4 and a C6 saccharified solution obtained by the solid-liquid separation 2 are concentrated in the concentration step. That is, in this embodiment, a dewatered cake 2 obtained by solid-liquid separation 2 is washed with water (water washing 2). By doing so, the dewatered cake 2 is reslurried to prepare a slurry 4. The slurry 4 is subjected to solid-liquid separation in the same manner as in the solid-liquid separation 2 and thus separated into washing water 2 and a dewatered cake 4 (solid-liquid separation 4). The present invention is characterized also in that the amount of C6 sugars remaining in the dewatered cake 2 is small. However, according to this embodiment, C6 sugars slightly remaining in the dewatered cake 2 can be recovered maximally by the water washing 2 and supplied to the fermentation step.

The washing water 2 in which C6 sugars are dissolved is mixed with a C6 saccharified solution obtained by the solid-liquid separation 2 and then concentrated by an RO membrane device so that the concentration of sugars is 10% by mass or higher. On the other hand, the dewatered cake 4 is appropriately disposed of outside the system.

The systems disclosed in FIGS. 2 and 3 may be combined to provide a system in which both the dewatered cake 1 and the dewatered cake 2 are washed with water and the washing water 1 and the washing water 2 are supplied to the concentration step to recover C5 and C6 sugars. In this case, it is possible to maximally recover both C5 sugars and C6 sugars to supply them to the fermentation step.

### Embodiment 4

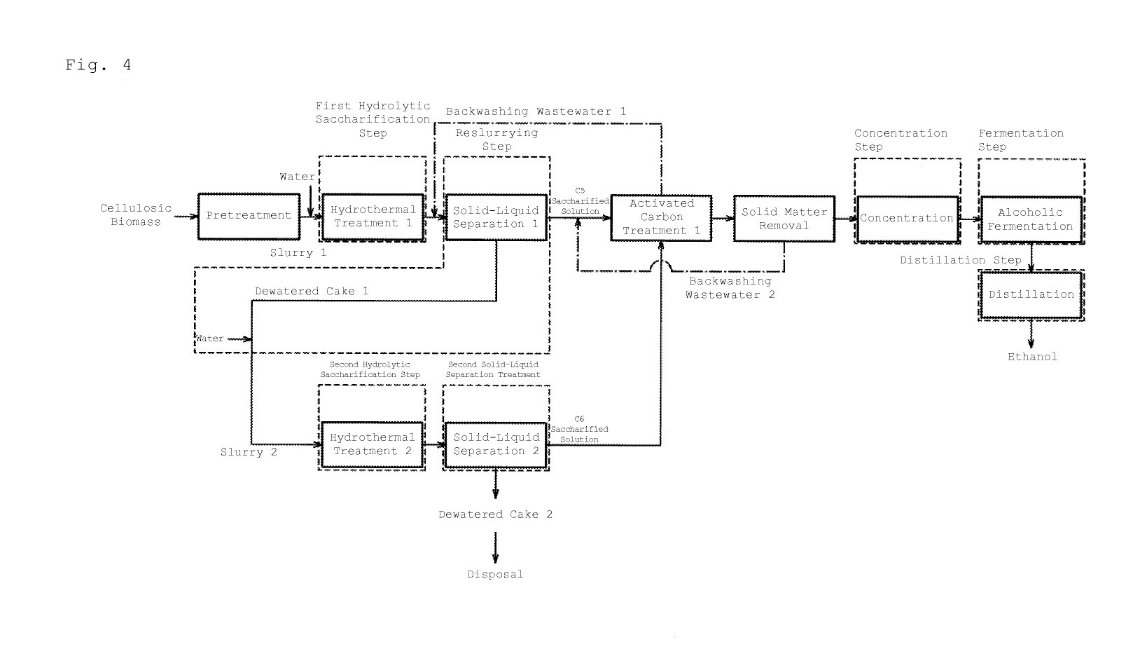

FIG. 4 shows a schematic flow chart illustrating Embodiment 4 of the present invention. A basic flow of this embodiment is the same as that of Embodiment 1, and therefore only the differences from Embodiment 1 will be described here. The same components as in Embodiment 1 are expressed by the same terms as used in Embodiment 1.

This embodiment is characterized in that a C5 saccharified solution obtained by solid-liquid separation 1 and a C6 saccharified solution obtained by solid-liquid separation 2 are subjected to activated carbon treatment before concentrated by an RO membrane device. The activated carbon treatment can be performed by, for example, supplying the saccharified solution to an activated carbon adsorption tower or a column packed with activated carbon. The activated carbon treatment of the saccharified solution makes it possible to remove an organic matter, such as lignin, or an inorganic deposit contained in the saccharified solution and therefore to prevent the clogging of an RO membrane of an RO membrane device used in the subsequent concentration step. The C5 saccharified solution and the C6 saccharified solution may be subjected to activated carbon treatment separately from each other, or may be mixed together and then subjected to activated carbon treatment.

The saccharified solution after activated carbon treatment is preferably subjected to a solid matter removal step to remove a solid matter upstream of an RO membrane device in order to prevent the clogging of an RO membrane of the RO membrane device with microparticles of activated carbon. Examples of a means for removing a solid matter, such as microparticles of activated carbon, from the saccharified solution after activated carbon treatment include, but are not limited to, an MF membrane device.

An activated carbon treatment means such as an activated carbon adsorption tower is preferably backwashed periodically. Backwashing wastewater 1 discharged during backwashing is supplied to the upstream side of a solid-liquid separation means used for the solid-liquid separation 1. Similarly, backwashing wastewater 2 discharged during backwashing of the MF membrane device is supplied to the upstream side of the activated carbon treatment means used for activated carbon treatment.

A system for activated carbon treatment and solid matter removal shown in FIG. 4 may be combined with Embodiments 1 to 3 shown in FIGS. 1 to 3.

### Embodiment 5

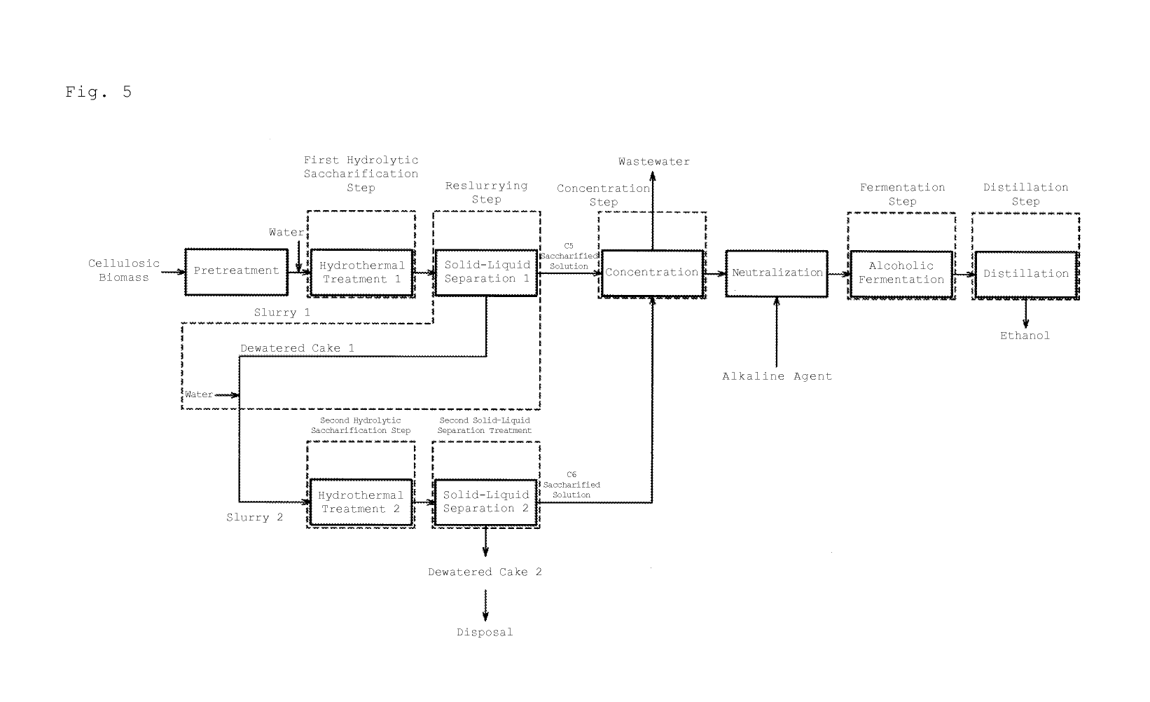

FIG. 5 shows a schematic flow chart illustrating Embodiment 5 of the present invention. A basic flow of this embodiment is the same as that of Embodiment 1, and therefore only the differences from Embodiment 1 will be described here. The same components as in Embodiment 1 are expressed by the same terms as used in Embodiment 1.

This embodiment is different from Embodiment 1 in that neutralization treatment is additionally performed before alcoholic fermentation to neutralize a concentrated saccharified solution obtained in the concentration step by adding an alkaline agent. As described above, the saccharified solution is often acidic with a pH of about 2 to 4. Therefore, when the saccharified solution is concentrated and directly subjected to the fermentation step, the pH of the saccharified solution is low and is not suitable for ethanol fermentation. Therefore, in this embodiment, the concentrated saccharified solution is neutralized by adding an alkaline agent to adjust its pH to about 4.0 to 6.0. The pH of the concentrated saccharified solution can be measured by a pH measuring device such as a pH meter.

The alkaline agent used for neutralization is not particularly limited as long as components contained in the saccharified solution are not decomposed or ethanol fermentation is not inhibited. However, from the viewpoint of ease of pH adjustment of the saccharified solution, a weak alkaline agent is more preferred than a strong alkaline agent. Specific examples of preferred alkaline agents include caustic soda and hydrated lime. The alkaline agent may be added as an aqueous solution, or may be added as a solid, such as a powder, as long as the alkaline agent is soluble in the saccharified solution.

From the foregoing explanations, many improvements and other embodiments of the present invention are apparent to a person skilled in the art. Therefore, the explanations above should be construed as illustrative examples provided for the purpose of teaching a person skilled in the art the best mode for carrying out the present invention. It is possible to substantially alter the details of the structure and/or functions without deviating from the spirit of the present invention.

### INDUSTRIAL APPLICABILITY

The ethanol production method according to the present invention is useful in the field of bioenergy as a method for producing ethanol by decomposing cellulosic biomass.

1. A method for producing ethanol using cellulosic biomass as a raw material, the method comprising:
a first hydrolytic saccharification step of hydrothermally treating a slurry of cellulosic biomass whose solid concentration is 1% by mass or higher but 5% by mass or lower at a temperature of 140° C. or higher but 200° C. or lower and a pressure of 1 MPa or higher but 5 MPa or lower to saccharify/decompose hemicellulose contained in the cellulosic biomass into C5 sugars;
a first solid-liquid separation step of subjecting the slurry after the first hydrolytic saccharification step to solid-liquid separation;
a reslurrying step of adding water to a dewatered cake obtained in the first solid-liquid separation step to prepare a slurry having a solid concentration of 1% by mass or higher but 5% by mass or lower;
a second hydrolytic saccharification step of hydrothermally treating the slurry obtained in the reslurrying step at a temperature of 240° C. or higher but 300° C. or lower and a pressure of 4 MPa or higher but 10 MPa or lower to saccharify/decompose cellulose contained in the cellulosic biomass into C6 sugars;
a second solid-liquid separation step of subjecting the slurry after the second hydrolytic saccharification step to solid-liquid separation;
a concentration step of concentrating a C5 saccharified solution obtained in the first solid-liquid separation step and a C6 saccharified solution obtained in the second solid-liquid separation step so that the concentration of sugars is 10% by mass or higher;
a fermentation step of subjecting a concentrated saccharified solution after the concentration step to alcoholic fermentation; and
a distillation step of distilling a fermented liquid obtained in the fermentation step to concentrate ethanol.

2. The ethanol production method according to claim 1, wherein the first solid-liquid separation step is a step of subjecting the slurry after the first hydrolytic saccharification step to solid-liquid separation and washing a resulting dewatered cake with water and then further subjecting the cake to solid-liquid separation, and wherein water separated after washing the dewatered cake with water in the first solid-liquid separation step is recovered and subjected to the concentration step.

3. The ethanol production method according to claim 1, wherein the second solid-liquid separation step is a step of subjecting the slurry after the second hydrolytic saccharification step to solid-liquid separation and washing a resulting dewatered cake with water and then further subjecting the cake to solid-liquid separation, and wherein water separated after washing the dewatered cake with water in the second solid-liquid separation step is recovered and subjected to the concentration step.

4. The ethanol production method according to claim 1, wherein before the concentration step, the C5 saccharified solution and the C6 saccharified solution are subjected to activated carbon adsorption treatment.

5. The ethanol production method according to claim 1, wherein the concentrated C5 saccharified solution and the concentrated C6 saccharified solution are subjected to neutralization treatment before the fermentation step.

6. The ethanol production method according to claim 2, wherein the second solid-liquid separation step is a step of subjecting the slurry after the second hydrolytic saccharification step to solid-liquid separation and washing a resulting dewatered cake with water and then further subjecting the cake to solid-liquid separation, and wherein water separated after washing the dewatered cake with water in the second solid-liquid separation step is recovered and subjected to the concentration step.
